(19) United States
(12) Patent Application Publication
(10) Pub. No.: US 2011/0159576 A1
(43) Pub. Date: Jun. 30, 2011

(54) Wastewater Biosolids Processing Methods

(76) Inventor: John Ralston Arnold, Clemont, FL (US)

(21) Appl. No.: 13/041,565

(22) Filed: Mar. 7, 2011

(51) Int. Cl. C12P 1/00

(52) U.S. Cl. 435/267

(57) ABSTRACT

A method to improve wastewater biosolids processing may include placing dried biosolids that have a moisture content lower than a moisture content needed for composting in a bioreactor. The method may also include processing the dried biosolids by controlling their temperature within the bioreactor while also reducing the moisture content of the dried biosolids. The method may further include removing the processed biosolids that have a moisture content lower than a moisture content needed for composting from the bioreactor.

### FIELD OF THE INVENTION

The invention relates generally to the field of wastewater biosolids, and particularly to the area of wastewater biosolids processing.

### BACKGROUND OF THE INVENTION

There are a number of systems to de-water wastewater sludge. For example, U.S. Pat. No. 7,181,865 to Kolega et al. '865 patent discloses a solar biosolids drying system that utilizes a mechanical tiller to reposition the deposited biosolids to aid in the drying process.

U.S. Pat. No. 7,335,311 to Christophersson also discloses a sludge treatment system and includes a press to dewater the biosolids, a shredder to reduce the size of the dewatered biosolids, and an auger to transport the shredding dewatered biosolids away from the system. Alternately, U.S. Pat. No. 7,175,683 to Cabello-Fuentes discloses a method for transforming liquid wastewater sludge into NPK-type granulated fertilizer. The method uses a filtration process combined with an exothermic reaction to reduce the water content in the sludge.

### SUMMARY OF THE INVENTION

A method to process wastewater biosolids may include placing dried biosolids that have a moisture content lower than a moisture content needed for composting in a bioreactor. The method may also include processing the dried biosolids by controlling their temperature within the bioreactor while also reducing the moisture content of the dried biosolids. The method may further include removing the processed biosolids that have moisture content lower than a moisture content needed for composting from the bioreactor.

The moisture content of the dried biosolids may be less than 40%, and/or the removed biosolids may be drier than the dried biosolids. The dried biosolids may not receive a bulking agent.

The biosolids' temperature may be controlled by the air flow in the rector, controlling parameters external to the bioreactor, and/or the rate of mixture within the bioreactor. The removed biosolids may comprise properly sized product that needs no screening process, no solid non-biosolids materials, and/or sizing of dust, flakes, granules, and/or granular composites.

The placing of the dried biosolids into the bioreactor may comprise a continuous feed operation and/or a batch feed operation. The processing may comprise reducing the pathogen count of the dried biosolids using temperature applied over time as required by the Environmental Protection Agency.

The method may also comprise drying the biosolids to a moisture content lower than a moisture content needed for composting. The drying may comprise distributing the biosolids on an outdoor drying pad in the sun, windrowing the biosolids, and/or raking the biosolids. The drying pad may comprise a highly solar absorbent surface and/or a semi-porous surface that aids in removing water content from the biosolids.

In one embodiment, the method may include drying biosolids to a moisture content lower than a moisture content needed for composting. The method may also include placing the dried biosolids in a bioreactor. The method may further include processing the dried biosolids by controlling their temperature within the bioreactor while also reducing the moisture content of the dried biosolids. The method may additionally include removing the processed biosolids that comprise properly sized product that needs no screening process, no solid non-biosolids materials, have a moisture content lower than a moisture content needed for composting, and/or sizing of dust, flakes, granules, and/or granular composites.

In one embodiment, the method may include placing dried biosolids that have a moisture content less that 40%, which is lower than a moisture content needed for composting, and the dried biosolids being devoid of any bulking agent. The method may also include processing the dried biosolids by controlling their temperature within the bioreactor while also reducing the moisture content of the dried biosolids. The method may further include removing the processed biosolids that have a moisture content lower than a moisture content needed for composting from the bioreactor, and the removed biosolids are drier than the dried biosolids.

### BRIEF DESCRIPTION OF THE DRAWINGS

FIG. 1 is a flowchart illustrating a method for processing bio-solids in accordance with the invention.

FIG. 2 is a flowchart illustrating method aspects according to the method of FIG. 1.

FIG. 3 is a flowchart illustrating another method for processing bio-solids in accordance with the invention.

FIG. 4 is a flowchart illustrating another method for processing bio-solids in accordance with the invention.

FIG. 5 is a graph of data gathered from the bioreactor during the processing in accordance with the invention.

### DETAILED DESCRIPTION OF THE PREFERRED EMBODIMENTS

The invention will now be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout. The invention is described below with reference to flowchart, illustrations, and/or block diagrams of methods, apparatuses, and systems according to exemplary embodiments of the invention.

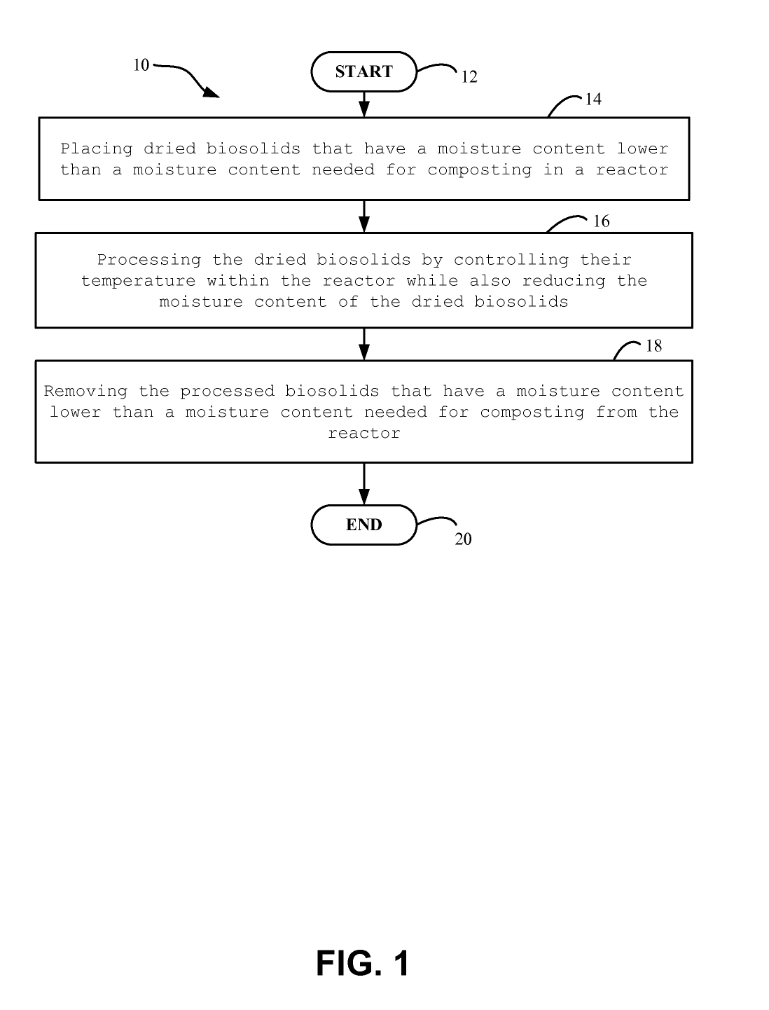

A method to process wastewater biosolids is now initially described with reference to flowchart 10 of FIG. 1. The method begins at Block 12 and includes placing dried biosolids that have a moisture content lower than a moisture content needed for composting in a bioreactor at Block 14, where composting is the process in which microorganisms break down organic matter when the correct combination of nitrogen, carbon, oxygen, and water are present. A bioreactor comprises a structure that supports an active biological environment as will be appreciated by those of skill in the art.

A proper water content for a composting process is in the range of 40%-60% as would be understood by those of skill in the art. Traditional composting requires the biosolids to have a minimum water content. The water content can be estimated by squeezing a handful of biosolids and it should have the feel of a well wrung sponge. If drier, water must be added. However, the method of FIG. 1 successfully utilizes a drier minimum water content whereby a squeezed handful of such biosolids will have a crunchy dry texture. In an embodiment, the moisture content of the dried biosolids are less than 40%.

In an embodiment, the method also includes processing the dried biosolids by controlling their temperature within the bioreactor while also reducing the moisture content of the dried biosolids at Block 16. In another embodiment, the biosolids' temperature is controlled by the air flow in the biorector, controlling parameters external to the bioreactor, and/or the rate of mixture within the bioreactor.

For example, air is either introduced or prevented from entering the bioreactor to regulate the biological activity taking place within the bioreactor thereby aiding in controlling the temperature within the bioreactor. Controlling the external parameters such as solar gain of the bioreactor, heat transfer between the bioreactor and the external environment, and/or the like also help to regulate the temperature within the bioreactor. The rate of biosolids mixing within the bioreactor also controls the rate of biological processes taking place within the bioreactor thereby aiding in controlling the temperature within the bioreactor.

The method further includes removing the processed biosolids that have a moisture content lower than a moisture content needed for composting from the bioreactor at Block 18. The method ends at Block 20. In other words, the processed biosolids are not compost because compost has a moisture content greater than 40%.

In an embodiment, the removed biosolids are drier than the dried biosolids. Stated another way, the processing continues to remove moisture content from the dried biosolids, which aids in the transportation of the removed biosolids because it is lighter than compost and therefore less costly to ship and therefore provides a smaller carbon footprint than compost. Removing moisture content from the removed biosolids also increases its value to buyers because purchasers are buying more product and less water weight.

In an embodiment, the dried biosolids do not receive and/or use a bulking agent, which current bioreactor biosolids processing requires. Current bioreactors use a 3 to 1 ratio of bulking agent to biosolids, which is an energy intensive process. The bulking agent for current bioreactor processes has to be collected, sized, screened, and transported to the bioreactor. As a result, the claimed method has a much smaller carbon footprint and lower operating cost than current bioreactor processes.

In an embodiment, the removed biosolids comprise properly sized product that needs no screening process, no solid non-biosolids materials, and/or sizing of dust, flakes, granules, and/or granular composites. Because no bulking agent was added, there are no solid non-biosolids materials in the removed biosolids and therefore no screening process is necessary related to the removal process, which again reduces the claimed method's carbon footprint.

Additionally, the no solid non-biosolids materials in the removed biosolids provides a consistent product for the end user who does not have to deal with clumps of uncomposted biosolids and/or chunks of uncomposted bulking agent. Further, the removed biosolids provides a fertilizer that is simpler to apply due to its consistent small size without any further processing thereby reducing the claimed method's carbon footprint.

In an embodiment, the dry nature of the claimed processing also limits the dried biosolids from clumping up thereby keeping the removed biosolids properly sized without additional steps. In another embodiment, the dried biosolids are compostable without a bulking agent because of their granular state, which enable the biosolids inside the bioreactor to have better aeration than a wetter and chunkier compost based material.

In an embodiment, the method also comprises drying the biosolids to a moisture content lower than a moisture content needed for composting. For example, the claimed method drying process is completed in 3-14 days whereas current drying processes are completed in 45-90 days. In another embodiment, the biosolids are dried to a moisture content lower than a moisture content needed for composting using applied heat/pressure.

In an embodiment, the drying comprises distributing the biosolids on an outdoor drying pad in the sun for solar and/or air drying. In one embodiment, the drying pad comprises a highly solar absorbent surface such as dark colored asphalt and/or the like. In another embodiment, the drying pad comprises a semi-porous surface that aids in removing water content from the biosolids. For example, a layer of asphalt can be applied to the top portion of the drying pad that has a specific porosity that will enable wet biosolids to drain faster than a non-porous surface.

In one embodiment, the biosolids are further dried on the drying pad by windrowing the biosolids and/or raking the biosolids. This manipulation of the biosolids on the drying pad causes the water content of the biosolids to drop thru evaporation, accelerated use by microorganisms within the biosolids, and/or the like. The windrowing and/or raking also helps in reducing the size of the biosolids to a more uniform sizing of dust, flakes, granules, and/or granular composites. The processing within the bioreactor also aids in sizing the removed biosolids.

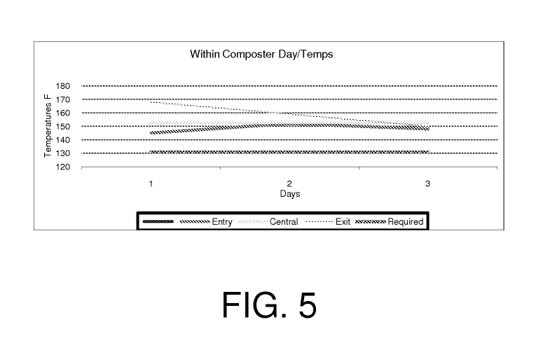

In an embodiment, the placing of the dried biosolids into the bioreactor comprises a continuous feed operation and/or a batch feed operation. With additional reference to FIG. 5, in another embodiment, the processing comprises reducing the pathogen count of the dried biosolids using temperature applied over time as required by the Environmental Protection Agency for a Class “AA” biosolids product. For instance, the claimed processing includes maintaining the temperature of the biosolids at 55° C. or higher for 3 days in the bioreactor thereby conforming to the pathogen reduction rules of the Environmental Protection Agency.

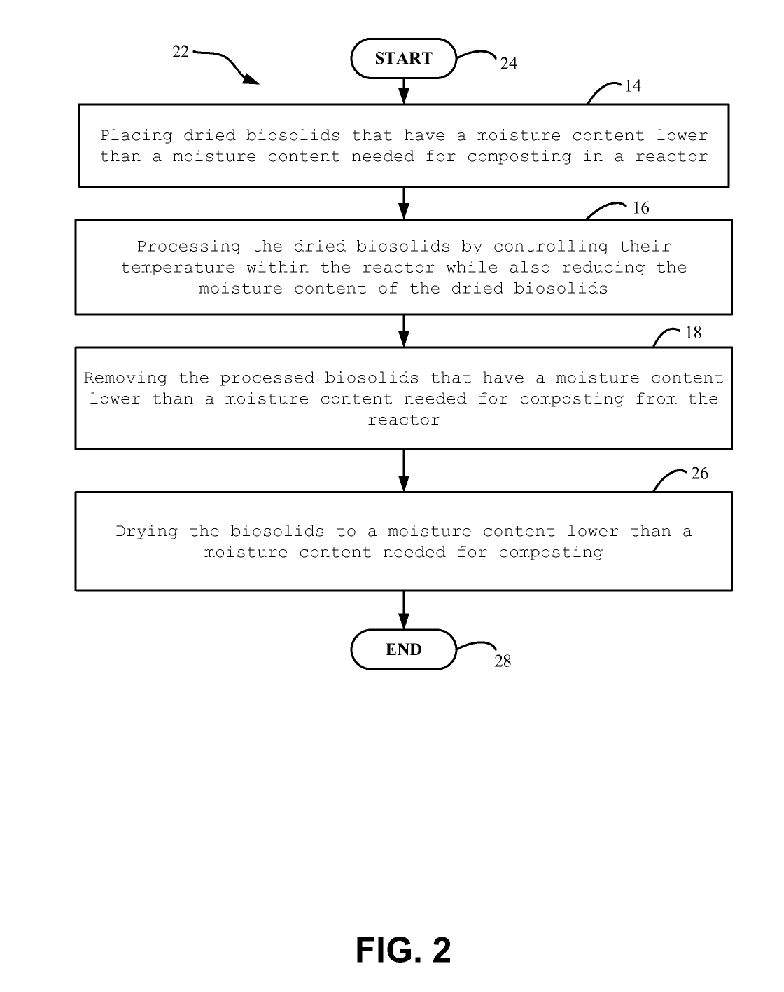

In another method embodiment, which is now described with reference to flowchart 22 of FIG. 2, the method begins at Block 24. The method includes the steps of FIG. 1 at Blocks 14, 16, and 18. The method may additionally include drying the biosolids to a moisture content lower than a moisture content needed for composting at Block 26. The method ends at Block 28.

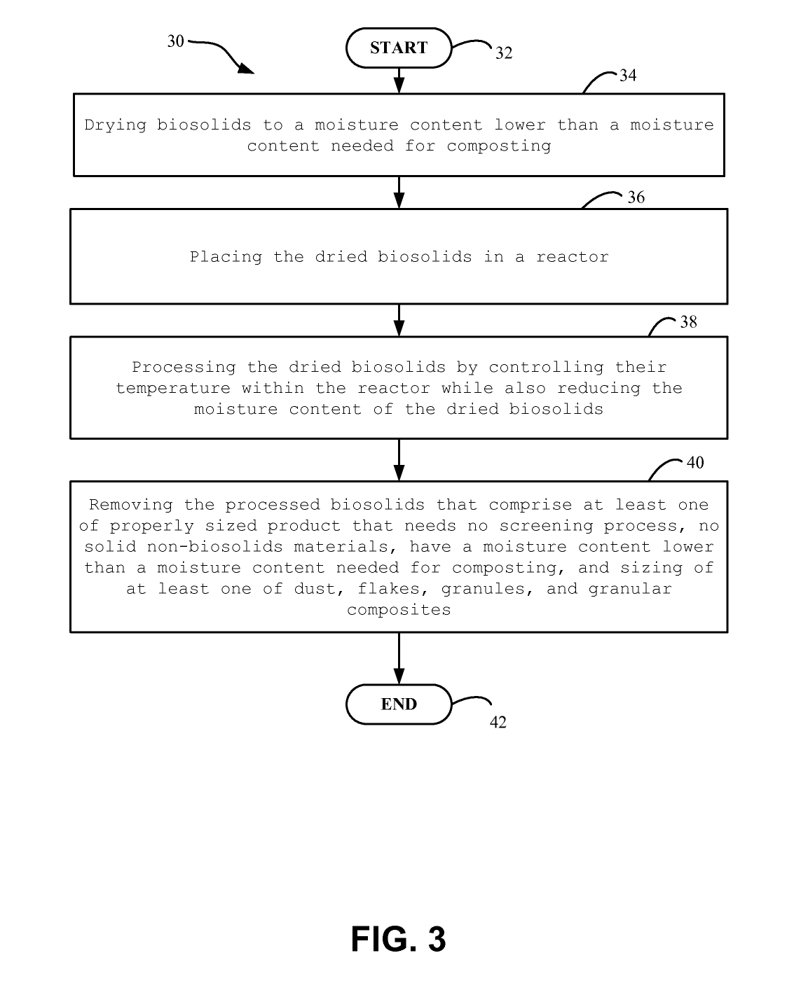

In another method embodiment, which is now described with reference to flowchart 30 of FIG. 3, the method begins at Block 32. The method includes drying biosolids to a moisture content lower than a moisture content needed for composting at Block 34. The method also includes placing the dried biosolids in a bioreactor at Block 36. The method may further include processing the dried biosolids by controlling their temperature within the bioreactor while also reducing the moisture content of the dried biosolids at Block 38. The method additionally includes removing the processed biosolids that comprise properly sized product that needs no screening process, no solid non-biosolids materials, have a moisture content lower than a moisture content needed for composting, and/or sizing of dust, flakes, granules, and/or granular composites at Block 40. The method ends at Block 42.

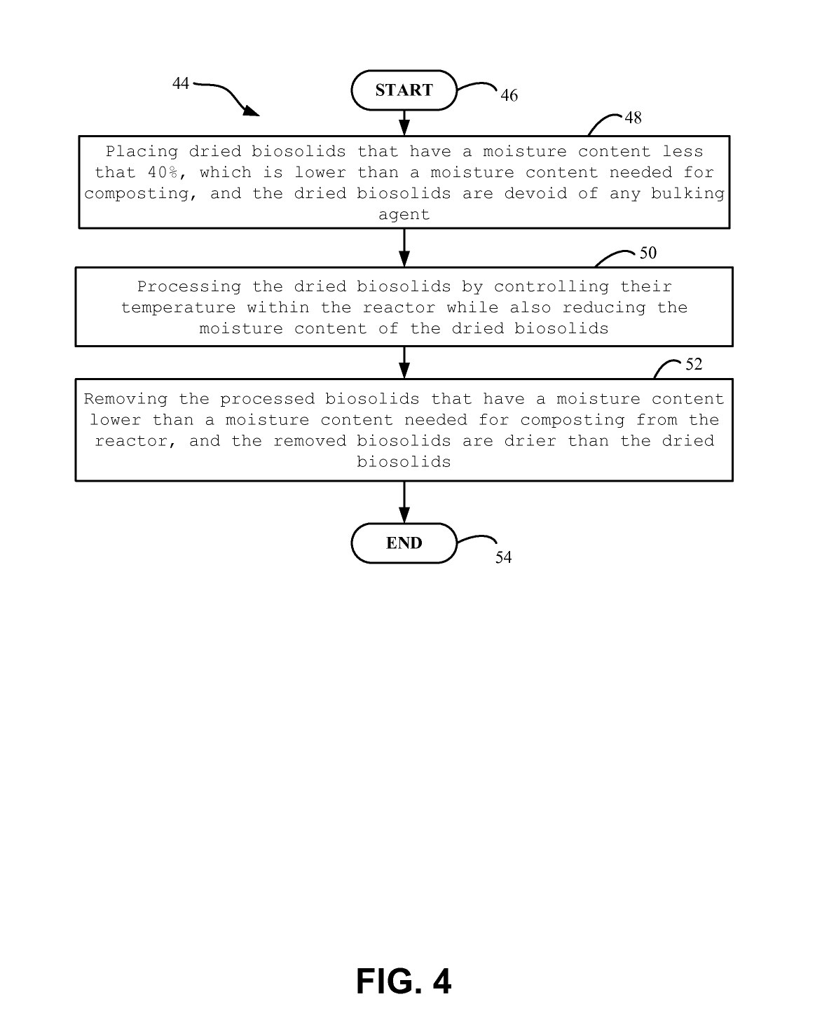

In another method embodiment, which is now described with reference to flowchart 44 of FIG. 4, the method begins at Block 46. The method includes placing dried biosolids that have a moisture content less that 40%, which is lower than a moisture content needed for composting, and the dried biosolids are devoid of any bulking agent at Block 48. The method also includes processing the dried biosolids by controlling their temperature within the bioreactor while also reducing the moisture content of the dried biosolids at Block 50. The method further includes removing the processed biosolids that have a moisture content lower than a moisture content needed for composting from the bioreactor, and the removed biosolids are drier than the dried biosolids at Block 52. The method ends at Block 54.

In view of the foregoing, the claimed methods convert biosolids to a more user friendly product, e.g. fertilizer, with a much smaller carbon footprint than any other current method. In addition, the claimed methods are much faster than a compost only system and much less resource intensive than a bioreactor only based system.

It should be noted that in some alternative implementations, the functions noted in a flowchart block may occur out of the order noted in the figures. For instance, two blocks shown in succession may, in fact, be executed substantially concurrently, or the blocks may sometimes be executed in the reverse order, depending upon the functionality involved because the flow diagrams depicted herein are just examples. There may be many variations to these diagrams or the steps (or operations) described therein without departing from the spirit of the invention. For example, the steps may be performed concurrently and/or in a different order, or steps may be added, deleted, and/or modified. All of these variations are considered a part of the claimed invention.

Furthermore, the use of the terms a, an, etc. do not denote a limitation of quantity, but rather denote the presence of at least one of the referenced item. The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that other modifications and embodiments are intended to be included within the scope of the appended claims.

That which is claimed is:

1. A method comprising:
placing dried biosolids that have a moisture content lower than a moisture content needed for composting in a bioreactor;
processing the dried biosolids by controlling their temperature within the bioreactor while also reducing the moisture content of the dried biosolids; and
removing the processed biosolids that have a moisture content lower than a moisture content needed for composting from the bioreactor.

2. The method of claim 1 wherein the moisture content of the dried biosolids is less than 40%, and the removed biosolids are drier than the dried biosolids.

3. The method of claim 1 wherein the dried biosolids do not receive a bulking agent.

4. The method of claim 1 wherein the biosolids' temperature is controlled by at least one of air flow in the rector, controlling parameters external to the bioreactor, and the rate of mixture within the bioreactor.

5. The method of claim 1 wherein the removed biosolids comprise at least one of properly sized product that needs no screening process, no solid non-biosolids materials, and sizing of at least one of dust, flakes, granules, and granular composites.

6. The method of claim 1 wherein the placing of the dried biosolids into the bioreactor comprises at least one of a continuous feed operation and a batch feed operation; and
wherein the processing comprises reducing the pathogen count of the dried biosolids using temperature applied over time as required by the Environmental Protection Agency.

7. The method of claim 1 further comprising drying the biosolids to a moisture content lower than a moisture content needed for composting.

8. The method of claim 7 wherein the drying comprises at least one of distributing the biosolids on an outdoor drying pad in the sun, windrowing the biosolids, and raking the biosolids, and the drying pad comprises at least one of a highly solar absorbent surface and a semi-porous surface that aids in removing water content from the biosolids.

9. A method comprising:
drying biosolids to a moisture content lower than a moisture content needed for composting;
placing the dried biosolids in a bioreactor;
processing the dried biosolids by controlling their temperature within the bioreactor while also reducing the moisture content of the dried biosolids; and
removing the processed biosolids that comprise at least one of properly sized product that needs no screening process, no solid non-biosolids materials, have a moisture content lower than a moisture content needed for composting, and sizing of at least one of dust, flakes, granules, and granular composites.

10. The method of claim 9 wherein the moisture content of the dried biosolids is less than 40%, and the removed biosolids are drier than the dried biosolids.

11. The method of claim 9 wherein the dried biosolids do not receive a bulking agent.

12. The method of claim 9 wherein the biosolids' temperature is controlled by at least one of air flow in the rector, controlling parameters external to the bioreactor, and the rate of mixture within the bioreactor.

13. The method of claim 9 wherein the placing of the dried biosolids into the bioreactor comprises at least one of a continuous feed operation and a batch feed operation; and
wherein the processing comprises reducing the pathogen count of the dried biosolids using temperature applied over time as required by the Environmental Protection Agency.

14. The method of claim 9 wherein the drying comprises at least one of distributing the biosolids on an outdoor drying pad in the sun, windrowing the biosolids, and raking the biosolids, and the drying pad comprises at least one of a highly solar absorbent surface and a semi-porous surface that aids in removing water content from the biosolids.

15. A method comprising:
placing dried biosolids that have a moisture content less that 40%, which is lower than a moisture content needed for composting, the dried biosolids being devoid of any bulking agent;
processing the dried biosolids by controlling their temperature within the bioreactor while also reducing the moisture content of the dried biosolids; and
removing the processed biosolids that have a moisture content lower than a moisture content needed for composting from the bioreactor, and the removed biosolids are drier than the dried biosolids.

16. The method of claim 15 wherein the biosolids' temperature is controlled by at least one of air flow in the rector, controlling parameters external to the bioreactor, and the rate of mixture within the bioreactor.

17. The method of claim 15 wherein the removed biosolids comprise at least one of properly sized product that needs no screening process, no solid non-biosolids materials, and sizing of at least one of dust, flakes, granules, and granular composites.

18. The method of claim 15 wherein the placing of the dried biosolids into the bioreactor comprises at least one of a continuous feed operation and a batch feed operation; and
wherein the processing comprises reducing the pathogen count of the dried biosolids using temperature applied over time as required by the Environmental Protection Agency.

19. The method of claim 15 further comprising drying biosolids to a moisture content lower than a moisture content needed for composting.

20. The method of claim 19 wherein the drying comprises at least one of distributing the biosolids on an outdoor drying pad in the sun, windrowing the biosolids, and raking the biosolids, and the drying pad comprises at least one of a highly solar absorbent surface and a semi-porous surface that aids in removing water content from the biosolids.
